(19) United States
(12) Patent Application Publication
(10) Pub. No.: US 2006/0269907 A1
(43) Pub. Date: Nov. 30, 2006

(54) Decontamination of biological fluids using diphenylpyrilium compounds

(75) Inventors: Stephen J Wagner, Columbia, MD (US); Andrey Skripchenko, Gaithersburg, MD (US)

Correspondence Address: BUCHANAN, INGERSOLL & ROONEY PC, POST OFFICE BOX 1404, ALEXANDRIA, VA, 22313-1404, US

(73) Assignee: American National Red Cross, Rockville, MD (US)

(21) Appl. No.: 10/558,560

(22) PCT Filed: Jun. 4, 2004
(86) PCT No.: PCT/US04/17625

§ 371 Date: Nov. 29, 2005

Related U.S. Application Data
(60) Provisional application No. 60/475,493, filed on Jun. 4, 2003.

(51) Int. Cl. A01N 1/02; A61K 31/33; A61K 31/382

(52) U.S. Cl. 435/2; 514/183; 514/432

(57) ABSTRACT

The present invention affords means for decontaminating biological fluids such as blood and blood components. The method involves contacting a biological fluid with a diphenylpyrilium compound, and irradiating the mixture with red light. The method is a potent and effective means for eliminating or diminishing active pathogens such as viruses, bacteria, and parasites, without causing substantial hemolysis or otherwise degrading the storage stability of the decontaminated biological fluid.

### REFERENCE TO PRIOR APPLICATION

This application claims the benefit of U.S. Provisional Application No. 60/475,493, filed Jun. 4, 2003.

### GOVERNMENT LICENSE RIGHTS

The U.S. Government has a paid-up license in this invention and the right in limited circumstances to require the patent owner to license others on reasonable terms as provided for by the terms of grant no. HL 66779 awarded by the NIH National Heart Lung and Blood Institute.

### BACKGROUND OF THE INVENTION

Over the past two to three decades, numerous donor screening and infectious disease testing methods have been implemented to reduce the transmission of human immunodeficiency virus (HW), hepatitis C virus (HCV), hepatitis B virus (OBV), and human T-cell lymphotropic viruses 1 and 2 (HTLV-1/2). As a result, the residual risk of virus transmission in blood components has declined to approximately 1 in 2,000,000 for HIV, 1 in 2,000,000 for HCV, 1 in 250,000 for hepatitis B virus, and 1 in 250,000 to 2,000,000 for HTLV-1 and 2. (Schreiber et al N Engl J Med 334:1685-1689; Dodd, Blood safety in the new millennium, Stramer ed, 1999, AABB press, Bethesda, Md.)

While the viral safety of the blood supply has dramatically increased with the adoption of donor screening and testing measures, the risk of bacterial sepsis and parasite infection has remained unchanged. Based on the available evidence, roughly 17% of transfusion related fatalities reported to the FDA between 1978 to 1998 were caused by bacterial contamination. These septic fatalities represent the greatest infectious disease risk among transfusion fatalities. (Lee, US FDA Bacterial Contamination of Platelets workshop, September, 1999) The risk is greatest from platelet transfusions, with 1 in 60,000 units (Ness et al., Transfusion 41:857-61 (2001)) to 1 in 450,000 units (Kuehnert et al., Transfusion 41:1493-9 (2001)) transfused resulting in fatalities. In addition, there is increasing recognition that parasites other than malaria, such as Babesia microti and Trypanosoma cruzi, can be transmitted by blood in the United States. (Dobroszycki et al., JAMA 28:927-30 (1999); Herwaldt et al Transfusion 42:1154-8 (2002); Kjemtrup et al Transfusion 42:1482-7 (2002); and Cimo et al., Tex Med 89:48-50 (1993)) Although there are less data available, the risk of transmission of these agents may be as great as or greater than those associated with the current viral risks for tested agents.

With the reduction of viral risk, the risks of sepsis and parasite infection have taken on increased relative importance. Development of pathogen reduction methods represents an approach to reduce transfusion-transmitted virus, bacteria, and parasite infections. In addition, pathogen inactivation may provide an additional layer of safety to reduce the residual risk from tested viruses, and may potentially reduce the transmission of unrecognized or uncharacterized blood borne agents.

Decontamination treatments that inactivate contaminating pathogens but do not harm the cellular fractions of blood either are not available or are impractical. Some decontamination treatments include the use of photosensitizers, which, in the presence of oxygen and upon exposure to light that includes wavelengths absorbed by the photosensitizer, inactivate viruses. (EP 0 196 515) Typically, such photochemicals are dyes or other compounds that readily absorb UV or visible light in the presence of oxygen. These compounds include merocyanine 540 (“MC540”) (U.S. Pat. No. 4,775,625), porphyrin derivatives (U.S. Pat. No. 4,878,891), phenothiazine derivatives (U.S. Pat. No. 6,030,767), as well as other photosensitizers.

Increased virucidal activity of these compounds is realized when the adsorption spectrum of the photosensitizer does not significantly overlap the absorption spectra of pigments present in the blood, such as hemoglobin. In order to minimize cellular damage, it is preferable that the photosensitizer be nontoxic to the cellular blood components and selectively bind to a component of the virus either that is not present in red cells or platelets, or, if present therein, that is not essential to red cells' or platelets' function. It is also preferable if the photodynamic treatment inactivates extracellular and intracellular viruses as well as proviruses. It is preferable if the photodynamic treatment inactivates bacteria and parasites as well. It is further preferable that the anti-pathogen activity of the photosensitizer is not significantly inhibited by the presence of plasma proteins, such as coagulation proteins, albumin, and the like.

Treatment with known photochemicals, however, frequently does damage to cellular blood components. For example, photochemicals such as the porphyrins (U.S. Pat. No. 4,878,891) and MC 540 (U.S. Pat. No., 4,775,625) cause cellular membrane damage in the presence of light and oxygen that significantly reduces the viability of the phototreated red cells during storage. Similarly, treatment of red blood cells using phthalocyanine 4 with type 1/type 2 quenchers caused red cell damage even under optimized conditions, ie., about 2% of the cells hemolyze after 21 days of storage. Current FDA guidelines recommend <1% hemolysis after 6 weeks of storage at 1-6° C. (Transfusion 35:367-70 (1995)) Finally, the phenothiazine, dimethylmethylene blue (DMMB), produces roughly 2% hemolysis following 6 weeks of 1-6° C. storage using a red cell storage solution, Erythrosol, that is designed to minimize or eliminate colloidal osmotic hemolysis (Transfusion 42:1200-1205 (2002)) With storage solutions that do not protect against colloidal osmotic hemolysis, such as ADSOL, roughly 25% of red blood cells hemolyze after 6 week refrigerated storage of red cell units treated with dimethylmethylene blue and light (Wagner et al, Transfusion 42:1200-1205 (2002))

Colloidal osmotic hemolysis arises from photodamage to the red cell membrane, which produces ion leakage, and results in an increased intracellular osmotic pressure at ionic equilibrium due to the osmotic pressure contribution from hemoglobin. This increased intracellular osmotic pressure induces water influx, and ultimately results in cell swelling and hemolysis. (Pooler, Biochim Biophys Acta 812:199-205 (1985))

Some residual hemolysis is still observed when red blood cells are treated with a photodynamic agent even when they are protected from colloidal osmotic hemolysis. (Wagner et al., Transfusion 42:1200-1205 (2002)) This non-colloidal osmotic hemolysis arises from two sources: from the photodynamic action of red blood cell membrane bound sensitizer, and from photodynamic action of unbound sensitizer. With dimethylmethylene blue for example, approximately 1.2% hemolysis still remains after six weeks of 1-6° C. storage even when phototreated red blood cells suspended in Erythrosol to minimize colloidal osmotic hemolysis are pre-incubated with a compound, quinacrine, that prevents dimethylmethylene blue binding to the red cell membrane. (Wagner et al., Photochem Photobiol 76:514-517 (2002))

Known photosensitizers thus induce hemolysis in red blood cells in several ways: 1) by producing membrane ion leaks, resulting in colloidal osmotic hemolysis 2) by inducing red blood cell membrane photodamage different from ion leaks which arise from membrane bound sensitizer, and 3) by generating red blood cell membrane photodamage different from ion leaks which arise from unbound sensitizer.

Solutions that prolong the shelf life of red cells are known. (e.g., U.S. Pat. No. 4,585,738) Typically, such solutions contain citrate, phosphate, glucose adenine, and other ingredients and function to prolong shelf life by maintaining the levels of ATP and 2,3-DPD in the cells. In addition, storage or additive solutions that contain high levels of the impermeable salt, citrate, can protect against colloidal hemolysis from phototreated red cells at ionic equilibrium by creating an extracellular osmotic pressure equal to the intracellular osmotic pressure arising from hemoglobin. (Transfusion 42:1200-1205 (2002)) Such storage solutions are known to those in the art and include ARC-8 and Erythrosol. (U.S. Pat. No. 4,585,738 and Vox Sang 65:271 (1993))

Certain antioxidants have been shown to protect cells from photoinduced hemolysis without greatly reducing the level of antiviral activity. However, none has been shown to be useful in the preparation of a transfusible product. For example, the red cell band 3 ligand, dipyridamole, partially protects red cells against dimethylmethylene blue photoinduced hemolysis by functioning as a red cell specific antioxidant (vanSteveninck et al., Transfusion 40:1330-1336 (2000) and Trannoy et al., Photochem Photobiol 75:167-171 (2002)) None of those methods have resulted in the desired level of protection from hemolysis (i.e., <1% hemolysis following 6 weeks of storage).

Despite techniques to minimize colloidal osmotic hemolysis by suspending red cells in a high citrate containing additive solution, by limiting membrane damage by preventing sensitizer binding to red cells through the use of a competitive binder, or by ameliorating photoinduced hemolysis by adding an antioxidant that specifically binds to red cell membrane proteins, no method or combination of methods has proved fully successful for decontaminating whole blood, blood components, or compositions containing concentrated blood components, including high levels of plasma. There remains, however, an acute need for a safe and effective method for reducing the level of active pathogenic contaminants, including viruses and bacteria, in whole blood or blood components without rendering the blood or blood components unsuitable for transfusion.

One of the limitations with traditional dyes acting as photosensitizers is that they produce active oxygen species whether or not they are bound to their target. Therefore unbound sensitizer can contribute to photoinduced collateral damage to the red cell membrane, leading to hemolysis. Therefore, development of a pathogen reduction method to limit photosensitization from unbound dye may be beneficial to red cell preservation.

Some dyes have the unusual properties of having a bond linking two aromatic conjugated double bond systems capable of rotation. Examples of these dyes include the methine bond of cyanine dyes and the carbon-carbon bonds in the 2′, 4′ and 6′ positions of pyrilium dyes. Many cyanine and pyrilium dyes are poor singlet oxygen photosensitizers because they can rotate about these carbon-carbon linkages, which reduces the lifetime of the dye's first excited singlet state, and limits both the fluorescent quantum yield and the potential for intersystem crossing over to the triplet state necessary for the generation of singlet oxygen. However, when these dyes are bound to a substrate with an orientation that facilitates a prolonged lifetime of the first excited state, enhanced fluorescence and enhanced singlet oxygen generation can occur. Therefore, pyrilium dyes can be used to either cause fluorescence or singlet oxygen mediated damage from bound dye without substantial contribution to fluorescence or singlet oxygen production from unbound dye. For example, pyrilium dyes have been used as nucleic acid fluorescent stains where the cell media or buffer does not have to be washed because little fluorescence is observed from unbound dye. (E.g., Nucleic Acids Symp 29:83-84 (1993); and U.S. Pat. No. 6,022,961) Pyrilium dyes have also been described as photosensitizers for the killing of cancer cells. (U.S. Pat. No. 6,242,477) These and all other patents and references cited herein are expressly incorporated by reference.

Despite the recognition that pyrilium dyes might be employed as photosensitizers, and observations that unbound pyrilium dyes are poor singlet oxygen generators, we are unaware of any proposal to use such compounds for pathogen reduction in biological fluids such as whole blood, blood components, or compositions containing concentrated blood components including high levels of plasma. In addition, one of ordinary skill in the art would not have been able to predict what class or kind of substituted pyrilium dyes could inactivate pathogens without otherwise deleteriously affecting the desired biochemical or physiological properties of a biological fluid, particularly blood, blood components, and plasma.

### SUMMARY OF THE INVENTION

The present invention provides methods for eliminating or diminishing active pathogenic contaminants, both intracellular and extracellular, in biological fluids without concomitant loss of desired biochemical or physiological properties of the fluid. In preferred embodiments, the biological fluids are selected from whole blood and blood components, including cellular blood components and liquid blood components. In particular, the present methods effect decontamination of biological fluids by eliminating or diminishing active pathogens such as viruses, bacteria (both gram negative and gram positive) and parasites without substantial hemolysis.

In one embodiment, the method involves decontaminating a biological fluid comprising the steps of: (a) adding a virucidal effective amount of a diphenylpyrilium compound to the biological fluid; and (b) irradiating the resulting mixture with red light for a time sufficient to eliminate or reduce the level of active pathogenic contaminants therein.

In preferred embodiments, the biological fluids are blood or blood components including cellular blood components, such as red blood cells (RBC_{S}) and platelets, and liquid blood components, such as plasma, or mixtures of cellular and/or liquid blood components.

As used herein, the term “pyrilium” refers to compounds having the general structure: 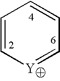
wherein Y is O, S, Se, or Te. Compounds useful in the present invention are pyrilium compounds substituted at the 2, 4, and 6 positions. Preferably, the compounds are diphenylpyrilium compounds.

The term “diphenylpyrilium compounds” or “diphenylpyrilium dyes” refers to 2,4-diphenylpyrilium compounds. Preferably, the phenyl substituents are further substituted at the para-position with one or more of the following substituents: alkyl, amino, alkylamino, alkoxyamino, aryl, arylamino, arylalkylamino, and arylalkoxyamino. Preferred compounds are those having the structure: 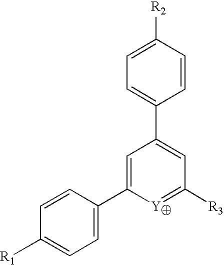
wherein:

Y is S, Se or Te;

R₁ and R₂ are independently selected from hydrogen, amino, alkylamino, aryl, arylamino, arylalkylamino, and arylalkoxyamino; and

R₃ is hydrogen, alkyl, alkoxy, alkylamino, aryl, arylamino, arylalkylamino, or arylalkoxyamino.

### BRIEF DESCRIPTION OF THE DRAWINGS

FIG. 1. Hemolysis during 1-6° C. storage of RBCs: Control RBCs stored in Erythrosol (RAS-2)(open triangles), Control RBCs stored in ADSOL (shaded triangles); RBCs stored in Erythrosol treated with compound 2 and red light (open squares); RBCs stored in ADSOL treated with compound 2 and red light (shaded squares); RBCs stored in Erythrosol (RAS-2) and containing dipyridamole (DPD) treated with compound 2 and red light (open circles); RBCs stored in ADSOL and containing dipyridamole treated with compound 2 and red light (shaded circles). The inset gives an expanded view of the same data over the 0 to 1% hemolysis range.

FIG. 2. Potassium release during 1-6° C. storage of RBCs: Control RBCs stored in Erythrosol (open triangles), Control RBCs stored in ADSOL (shaded triangles); RBCs stored in Erythrosol treated with compound 2 and red light (open squares); RBCs stored in ADSOL treated with compound 2 and red light (shaded squares); RBCs stored in Erythrosol and containing dipyridamole treated with compound 2 and red light (open circles); RBCs stored in ADSOL and containing dipyridamole treated with compound 2 and red light (shaded circles).

FIG. 3 illustrates the structures of thiopyrilium (TP), or 2′,4′-bis(4-N,N-dimethylaminophenyl) 6′-methylthiopyrylium iodide, and diphenylpyrilium (DP).

FIG. 4 illustrates the results from a spectroscopic assay measuring the effect of DP on TP binding to RBCs suspended in Erythrosol.

FIG. 5 shows the log₁₀ inactivation of virus as a function of TP concentration.

FIG. 6 shows the effect of dipyridamole and choice of additive solution on hemolysis following the storage of phototreated RBCs (160 μM TP and 1.1 J/cm² light).

FIG. 7 shows the effect of 160 μM TP, 200 μM DP and 1.1 J/cm² light on morphology score, pH, glucose utilization, lactate production, and ATP levels of RBCs suspended in Erythrosol in panels A through E, respectively.

FIG. 8 shows the effect of DP on potassium release from RBCs suspended in Erythrosol and treated with 160 μM TP and 1.1 J/cm² light.

### DETAILED DESCRIPTION OF THE INVENTION

The present invention provides methods for decontaminating a biological fluid comprising: (a) contacting a biological fluid with a decontamination effective amount of a diphenylpyrilium compound; and (b) irradiating the resulting mixture with light of about 560 to about 800 nm to achieve a decontaminating effect.

In a preferred embodiment, the invention provides a method for decontaminating a biological fluid comprising: (a) adding 2′,4′-bis(4-N,N-dimethylaminophenyl) 6′-methylthiopyrilium iodide to the biological fluid to a concentration of about 100 to about 300 μM; (b) adding dipyridamole to the biological fluid to a concentration of about 100 to about 300 μM; and (c) irradiating the resulting biological fluid with light of about 560 to about 800 nm.

The term “decontamination” means both a process whereby the level of active pathogen actually present in a given composition is eliminated or reduced, and a process for assuring that a potential pathogenic contaminant within a composition is below a certain level regardless whether such contaminant was ever present in the composition. Decontamination can be effected by rendering pathogens inactive and/or noninfectious or by reducing the number of pathogens in the composition. A composition containing whole blood or a blood component that has been “decontaminated” can be transfused or manipulated without harming or infecting anyone exposed thereto.

Preferably, the level of decontamination achieved will be such that the immune system of the organism exposed to or transfused with the biological fluid will be capable of overcoming the pathogenic effect thereof, and preventing the onset of any disease associated therewith. It will be understood that, as so defined, the level of decontamination will vary depending upon the pathogen.

A decontamination-effective amount of a diphenylpyrilium compound is also referred to herein as a virucidal effective amount. A decontamination- or virucidal-effective amount is that capable of achieving a statistically significant reduction in the level of active pathogenic virus in the biological fluid. Preferably, the virucidal effective amount is that capable of achieving reduction of at least about 4.0 log₁₀ extracellular VSV (Vesicular Stomatitis Virus) in blood or blood components. More preferably, it is that capable of achieving at least about 5.0 log₁₀ extracellular VSV reduction and at least about 2.5 log₁₀ intracellular VSV reduction. Still more preferably, it is that capable of achieving at least about 7.0 log₁₀ extracellular VSV reduction and at least about 5.0 log₁₀ intracellular VSV reduction. It will be understood that the amount of diphenylpyrilium compound necessary to achieve the desired decontamination will vary from compound to compound, but that the means for assaying the virucidal effect of the various diphenylpyrilium compounds embraced by the claims is well within the skill level of one of ordinary skill in the art.

The terms “decontamination effective amount” and “virucidal effective amount” also mean an amount sufficient to provide a concentration of diphenylpyrilium compound in the biological fluid that is both acceptable for transfusion and is effective in reducing the level of active pathogens in the composition when irradiated with light of an appropriate intensity and wavelength.

As discussed more filly below, the effective concentration of diphenylpyrilium compound to be used can be determined empirically by one of ordinary skill in the art. In preferred embodiments, the effective concentration of diphenylpyrilium compound is about 50 to 300 μM, and more preferably 100 to 200 μM.

Preferably, the diphenylpyrilium compound is non-toxic, and the effective concentration is acceptable for transfusion so that the biological fluid does not require additional manipulation to remove the diphenylpyrilium compound and thereby risk contamination. Alternatively, the diphenylpyrilium compound concentration in the decontaminated biological fluid can be reduced by washing or by adsorption to some biologically compatible resin.

It will be likewise be understood that such virucidal effective amounts will be effective in achieving statistically significant reductions in active pathogens other than viruses. As used herein, the term “pathogen” or “pathogenic contaminant” means a contaminant that, upon handling or transfusion into a recipient is capable of causing disease in the handler and/or recipient. Examples of pathogenic contaminants include, but are not limited to: viruses, such as retroviruses (e.g. HIV) and hepatitis viruses; bacteria, such as E. coli; parasites, such as Trypanosoma; and leukocytes, such as lymphocytes (which can be a reservoir for harboring intracellular viruses).

The term “pathogen” also includes any replicable agent that rnay be found in or infect whole blood or blood components. Such pathogens include the various viruses, bacteria, parasites, and leukocytes known to those skilled in the art to generally be found in or infect whole blood or blood components. Illustrative examples of such pathogens include, but are not limited to: bacteria, such as Streptococcus species, Escherichia species, and Bacillus species, viruses, such as human immunodeficiency viruses and other retroviruses, herpes viruses, paramyxoviruses, cytomegaloviruses, hepatitis viruses (including hepatitis B and hepatitis C), pox viruses, and toga viruses; parasites, such as malarial parasites, including Plasmodium species, and trypanosomal parasites; and leukocytes, such as lymphocytes. The methods of the present invention afford means for achieving pathogen inactivation corresponding to at least about 6 log₁₀ of bacteria and other pathogens.

As used herein, the term “biological fluid” means fluids of biological significance or origin including blood or mixtures or suspensions comprising blood components, milk, tears, saliva, urine, cell culture supernatants, cell extracts, and cellular supernatant. Preferably, the biological fluid is blood or blood components. Unless stated otherwise, “blood” refers to mammalian blood.

The term “blood components” means one or more of the constituent components of blood that can be separated from whole blood. The term includes cellular blood components, such as red blood cells and platelets; blood proteins, such as blood clotting factors, enzymes, albumin, plasminogen, and immunoglobulins; and liquid blood components, such as plasma and plasma-containing compositions, and mixtures containing plasma derivatives and/or plasma proteins.

The term “cellular blood component” means one or more components of whole blood that comprises cells, such as red blood cells or platelets.

The term “blood protein” means one or more proteins normally found in whole blood. Illustrative examples of blood proteins found in mammals (including humans) include, but are not limited to, coagulation proteins (both vitamin K-dependent, such as Factor VII or Factor IX, and non-vitamin K-dependent, such as Factor VIII and von Willebrands factor), albumin, lipoproteins (high density lipoproteins and/or low density lipoproteins), complement proteins, globulins (such as immunoglobulins IgA, IgM, IgG and IgE), and the like.

As used herein, the term “liquid blood component” is intended to mean one or more of the fluid, non-cellular components of whole blood, such as plasma (the fluid, non-cellular portion of blood of humans or animals as found prior to coagulation), or serum (the fluid, non-cellular portion of the blood of humans or animals after coagulation).

The term “composition containing the cellular blood component and/or a blood protein” is intended to mean a composition that contains a biologically compatible solution, such as ARC-8 or Erythrosol, and one or more cellular blood components, one or more blood proteins, or a mixture of one or more cellular blood components and/or one or more blood proteins. Such compositions may also contain a liquid blood component, such as plasma.

The biological fluids to be decontaminated according to the methods of the present invention can be leukodepleted. The term “leukodepleted” means that the concentration of leukocytes in the composition has been reduced by a specified amount, such as a factor of 10⁵. In preferred embodiments, the biological fluids to be decontaminated in accordance with the present invention will be first leukodepleted.

The phrase a “transfusible composition” means a composition that can be transfused into the blood stream of a mammal. Transfusible compositions might be whole blood or otherwise contain one or more blood components, such as one or more cellular blood components, one or more blood proteins, and one or more liquid blood components; or mixtures of whole blood and one or more blood components, such as red blood cells, clotting factors, or plasma.

The ratio of the titer of the control sample to the titer of virus in each of the treated samples is a measure of viral inactivation. As used herein, the term “log₁₀ inactivation” is intended to mean the log₁₀ of this ratio. Typically, a log₁₀ inactivation of at least about 4 indicates that the treated sample has been decontaminated.

The term “fluence” means a measure of the energy per unit area of sample and is typically measured in joules/cm² (J/cm²). As used herein, the term “fluence rate” is intended to mean a measure of the amount of energy that strikes a given area of a sample in a given period of time and is typically measured as milliwatts (mW)/cm² or joules/Cm² per unit of exposure.

As used herein, the term “diphenylpyrilium dye” or “diphenylpyrilium compound” means a compound having the general structure: 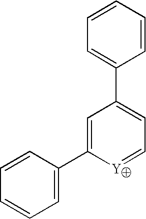
When employed in the methods of the present invention, the diphenylpyrilium compound will have one or more amino substituents on one or more of the phenyl groups. Preferred compounds are soluble in polar solvents, particularly aqueous solvents, and are capable of passing through the cell membrane of blood cells in sufficient quantity to reduce the level of active intracellular pathogenic contaminants upon irradiation with light of a suitable intensity and wavelength without causing unacceptable levels of hemolysis. Preferably, the method achieves the desired decontamination with less than about 5% hemolysis. More preferably, hemolysis resulting from the practice of the present method is less than about 3%, and still more preferably less than about 1%.

The unspecified valences of the carbon atoms in the formula above can be occupied by hydrogen or by any organic or inorganic moiety that does not adversely affect the amphiphilic character of the diphenylpyrilium compound.

One skilled in the art can determine the suitability of a particular substituent group or groups empirically using standardized assays for determining the level of active intracellular and extracellular pathogenic contaminants and standardized assays for determining hemolysis levels. As used herein, hemolysis is measured after 42 days storage at 1-6° C.

Illustrative examples of suitable substituents include, but are not limited to, alkyl groups, alkenyl groups, allynyl groups, hydroxyl groups, alkoxy groups, aryl groups, heteroaryl groups, aryloxy groups, heteroaryloxy groups, nitro groups, amine groups, amide groups, alkylcarboxyl groups, arylhaloalkyl groups haloaryl groups. Preferred organic moieties include alkyl groups, such as methyl, ethyl and propyl; alkenyl groups such as ethenyl; alkynyl groups such as acetenyl; and amines such as methylamine and dimethylamine.

The term “leukocyte depleted blood component” is intended to mean a blood component, such as plasma, as defined above that has been filtered through a filter that depletes the concentration of leukocytes in the plasma by a factor as least 10³. Such filters are identified by the log of the factor by which the blood component is depleted of leukocytes.

The term “extracellular pH” means the pH of the liquid medium in which cellular blood components, such as red blood cells, are stored or maintained.

The term “a biologically compatible solution” is intended to mean an aqueous solution to which cellular blood components can be exposed, such as by being suspended therein, and remain viable, i.e., retain their essential biological and physiological characteristics.

Preferably, such biologically compatible solutions contain an effective amount of at least one anticoagulant. Preferred biologically compatible solutions in the context of this invention protect against colloidal osmotic photoinduced hemolysis. One method for achieving this is by the addition of citrate at concentrations that balance the osmotic pressure contributed by hemoglobin.

The term “a biologically compatible buffered solution” is intended to mean a biologically compatible solution having a pH and osmotic properties (e.g., tonicity, osmolality and/or oncotic pressure) suitable for maintaining the integrity of the cell membrane of cellular blood components. Suitable biologically compatible buffered solutions typically have a pH between 5 and 8.5 and are isotonic or only moderately hypotonic or hypertonic. Biologically compatible buffered solutions are known and readily available to those of skill in the art. Illustrative examples of suitable solutions include, but are not limited to, those listed in Table 1 below showing the substances present in anticoagulant solution into which whole blood is drawn, and the substances present in the additive solution added after whole blood is centrifuged and plasma removed to make packed red cells. Additive solutions containing citrate such as Nutricell and Erythrosol are preferred because these solutions protect against 10 colloidal osmotic hemolysis, whereas those lacking citrate such as ADSOL do not.

TABLE 1

| Anticoag. (70 mL) | CPD¹ | CP2D | CPD |
|---|---|---|---|
| Dextrose (hydrous) | 1.785 g | 3.57 g | 1.785 g |
| Sodium citrate | 1.84 g | 1.84 g | 1.84 g |
| Citric acid | 0.229 g | 0.229 g | 0.229 g |
| Sodium phosphate | 0.155 g | 0.155 g | 0.155 g |
| monobasic (dihydrate) | | | |

| Additive soln (110 mL) | ADSOL | Nutricell | Erythrosol² |
|---|---|---|---|
| Dextrose (hydrous) | 2.42 g | 1.21 g | 0.99 g |
| Sodium chloride | 0.990 g | 0.451 g | 0.860 g |
| Adenine | 0.0297 g | 0.033 g | 0.0237 g |
| Mannitol | 0.825 g | — | 0.851 g |
| Sodium citrate (dihydrate) | — | 0.647 g | 0.86 g |
| Citric acid | — | 0.0462 g | — |
| Sodium phosphate | — | 0.304 g | 0.0711 g |
| monobasic (dihydrate) | | | |
| Sodium phosphate dibasic | — | — | 0.257 g |
| (dihydrate) | | | |

¹CPD = citrate, phosphate, dextrose; CP2D = CPD having twice the concentration of dextrose.
²Also referred to as RAS-2 (Red Cell Additive Solution No.2).

In certain preferred embodiments, whole blood is first drawn from a donor into a suitable biologically compatible buffered solution containing an effective amount of at least one anticoagulant. Suitable anticoagulants are known to those skilled in the art, and include, but are not limited to, lithium, potassium or sodium oxalate (15 to 25 mg/10 mL of blood), sodium citrate (40 to 60 mg/10 mL blood), heparin sodium (2 mg/10 ml of blood), disodium EDTA (10 to 30 mg/10 mL of blood) or ACD-Formula B solution (1.0 mL/10 mL blood).

The whole blood so collected can be decontaminated according to the methods of the present invention. Alternatively, the whole blood can be separated into blood components, including, but not limited to plasma, platelets and red blood cells, by any method known to those of skill in the art. For example, blood can be centrifuged for a sufficient time and at a sufficient centrifugal force to sediment the red blood cells. Leukocytes collect primarily at the interface of the red cells and the plasma-containing supernatant in the buffy coat region. The supernatant, which contains plasma, platelets, and other blood components, can be removed and centrifuged at a higher centrifugal force, whereby the platelets sediment.

Human blood normally contains about 3×10⁹ leukocytes per 500 mL of whole blood (1 unit). The concentration of leukocytes, which sediment with the red cells, can be decreased if desired by passing through a filter that decreases leukocyte concentration by selected orders of magnitude. Leukocytes can also be removed from each of the components by filtration through an appropriate filter that removes them from the solution.

In one preferred embodiment, the whole blood or blood component to be decontaminated is obtained in, prepared in, or introduced into, gas permeable blood preservation bags, which are sealed and flattened to a width sufficiently narrow to permit light to irradiate the contents, such that any pathogenic contaminant present in the blood or blood component in the bag will be irradiated. Conventional blood bags used in the art can be used provided the bag is transparent to the selected wavelength of light.

In an alternative preferred embodiment, blood can be passed from one bag through tubing into another bag, which serves as a flow cell, and is flattened to a width sufficiently narrow to permit light to irradiate the flow cell contents, such that any pathogenic contaminant present in the blood or blood component in the bag will be irradiated, and the irradiated blood is subsequently passed through tubing into a receiving blood bag.

Optionally, the gas permeable blood preservation bag also contains oxygen. While not wishing to be bound by any theory of operability, it is believed that certain species of amphiphilic diphenylpyrilium compound employed in the methods of the invention, in addition to intercalating between base pairs of DNA, generate singlet oxygen when irradiated with light of an appropriate wavelength. As is known to those skilled in the art, singlet oxygen directly or products thereof (e.g., superoxides, hydroxy radicals, etc.) cause pathogen inactivation. Accordingly, it is preferred that, at least for certain species of amphiphilic diphenylpyrilium compounds, the composition being decontaminated contain a suitable amount of oxygen.

The composition that is to be decontaminated may also include any suitable biologically compatible buffer known to those of skill in the art. Examples of such buffers include, but are not limited to, AC2D/Nutricell and ACD/Erythrosol. In a preferred embodiment of this invention, the biologically compatible buffer is ACD/Erythrosol.

The irradiation step can be performed in any fashion that ensures that the diphenylpyrilium compound is thoroughly distributed throughout the biological fluid and is exposed to sufficient light to achieve the desired decontamination effect. Preferably, the irradiation step is performed on a thin layer or film of the biological fluid. Alternatively, the irradiation step can be performed on the biological fluid in a conventional vessel with appropriate stirring or agitation to effect thorough irradiation throughout the mixture. One of ordinary skill in the art will appreciate that such alternative embodiments might require light of greater energy to effect the desired level of irradiation.

Although it will be understood that the parameters can be varied, exemplary irradiation conditions are those wherein the thin film is of a thickness of about 0.5 mm to about 3 mm, and more preferably about 1 mm. The film is irradiated with light of wavelength of about 560 to about 800 nm, preferably about 590 to about 640 nm; and still more preferably about 620 nm. One of ordinary skill in the art will appreciate that as one deviates from light of the optimal wavelength, greater amounts of energy may be required to achieve the same decontamination effect.

Irradiation of sufficient energy is effected to achieve the desired level of decontamination. Generally, irradiation of at least about 0.025 j/cm² of light of about 560 to about 800 nm is effected; and preferably, irradiation of about 0.05 to about 5.0 J/cm² of light of about 560 to about 800 nm is effected. More preferably, irradiation of about 0.1 to about 1.0 j/cm² of light of about 590 to about 640 nm is effected. Still more preferably irradiation of about 0.1 to about 0.4 J/cm² of 620 nm light (which corresponds to about 1.1 to about 2.2 J/cm² of 670 nm light) is effected.

The preferred amphiphilic diphenylpyrilium compounds employed in the methods of the present invention include those of the formula: 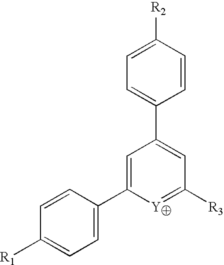
wherein: each of R₁, R₂, and R₃ is independently selected from the group consisting of an alkyl group, an alkenyl group, an alkynyl group, an alkoxy group, hydroxyl, amino, alkylamino, aryl, arylamino, arylalkylamino, arylalkoxyamino (e.g., phenylmorpholino) and hydrogen; and Y is sulfur or selenium. In preferred embodiments, Y is sulfur or selenium; R₁ and R₂ are independently selected from hydrogen, amino, alkylamino(monalkylamino and dialkylamino) and alkoxyamino (including heterocycles incorporating oxygen and/or nitrogen within the ring, e.g., morpholino); and R₃ is hydrogen, alkyl, alkoxy, aryl, arylamino, arylalkylamino, or arylalkoxyamino.

The term “alkyl group” means a straight or branched chain hydrocarbon radical having from 1-10 carbon atoms; preferably 1 to 6 carbon atoms; and more preferably 1 or 2 carbon atoms.

The term “alkenyl group” means a straight or branched chain hydrocarbon radical having 2-10 carbon atoms and at least one carbon-carbon double bond.

The term “alkynyl group” means a straight or branched chain hydrocarbon radical having 2-10 carbon atoms and at least one carbon-carbon triple bond.

The term “axyl group” means a cyclic aromatic hydrocarbon radical having from 6-12 carbon atoms; preferably 6-10 carbon atoms; and includes groups such as phenyl, naphthyl, and the like.

The term “aralkyl group” means a straight or branched chain hydrocarbon radical having from 1 to 6 carbon atoms bound to a cyclic aromatic hydrocarbon radical having from 6-12 carbon atoms in the ring(s), and includes radicals such as benzyl, 2-phenylethyl and the like.

The term “heteroaryl group” is intended to mean a monocyclic or bicyclic aromatic radical having from 4-11 carbon atoms and at least one heteroatom (i.e. an oxygen atom, a nitrogen atom and/or a sulfur atom) in the ring(s), such as thienyl, fulryl, pyranyl, pyridyl, quinolyl and the like.

In a preferred embodiment, R₁ and R₂ are independently selected from the group consisting of: amino, monomethylamino or dimethylamino. In a more preferred embodiment, R₁ and R₂ are both dimethylamino.

In a preferred embodiment, R₃ is alkyl, alkoxy, or aryl. In a more preferred embodiment, R₃ is alkyl of 1-6 carbons or phenyl. In a still more preferred embodiment, R₃ is methyl. The term alkoxy refers to an alkyl ether wherein the alkyl group is as defined above.

The amphiphilic diphenylpyrilium compounds used in the methods of the present invention can be prepared according to methods and techniques known to those of ordinary skilled in the art. Suitable synthetic methods for the preferred compound are described, for example, in U.S. Pat. No. 6,022,961, which is incorporated herein by reference.

In a particularly preferred embodiment of the present invention, 2′,4′-bis(4-N,N-dimethylaminophenyl) 6′-methylthiopyrilium iodide 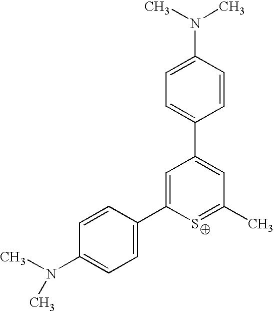
is employed as the amphiphilic diphenylpyrilium dye. Preferably, the 2′,4′-bis(4-N,N-dimethylaminophenyl) 6′-methylthiopyrilium iodide is introduced into the whole blood or blood component to be decontaminated at a concentration of about 100 to 200 μM.

The mixture of the whole blood and/or blood components and amphiphilic diphenylpyrilium compound is then irradiated with light of an appropriate wavelength (or a mixture of wavelengths) and intensity. As used herein, the term “appropriate wavelength and intensity” is intended to mean light of a wavelength and intensity that can be absorbed by the diphenylpyrilium compound, but does not damage the blood or blood components present. It is within the level of ordinary skill in the art to select such wavelength and intensity empirically based on certain relevant parameters, such as the particular compound employed and its concentration in the composition. For example, one having skill in the art would appreciate that if the intensity of the light source is decreased, a greater concentration of diphenylpyrilium compound and/or longer exposure time could offset the decrease in intensity. Likewise, the use of light of less optimal wavelength can be offset by increasing the radiant energy.

An appropriate wavelength is preferably selected based on the absorption profile of the diphenylpyrilium compound employed, and is most preferably one that does not result in substantial damage to one or more of the cellular blood components in the composition being decontaminated.

Known model viral systems can be used to test the selected dye and the light source for efficacy. Model viral systems include, but are not limited to, vesicular stomatitis virus (“VSV”: an animal virus the genome of which is encoded in single stranded RNA), and Pseudorabies virus (an animal virus that contains its genome in double stranded DNA). Based on the effective values of parameters such as wavelength and light intensity measured for such model systems, one of skill in the art can routinely select suitable values for these parameters for use in practice of the present invention.

In a preferred embodiment of this invention, oxygenated red blood cells, which have been leukodepleted with a five log filter, are first suspended in Erythrosol or Nutricell at a hematocrit of about 15 to about 50 percent, dipyridamole is added at a final concentration of about 50 to 300 μM, and 2′,4′-bis(4N,N-dimethylaminophenyl) 6-methylthiopyrilium iodide is added to a final concentration of about 100 to 200 μM. The blood is placed in a flattened container to produce a thin film. Preferably, the thin film is of a thickness of about 0.5 mm to about 3 mm, and more preferably about 1 mm. This film is irradiated with red light of wavelength of about 560 to about 800 nm at sufficient energy to reduce the level of active pathogenic contaminant in the blood.

In other embodiments of this invention, biological fluids containing platelets and fluids containing high concentrations of plasma can be decontaminated by contact with an effective amount of an amphiphilic diphenylpyrilium compound for sufficient time plus irradiation with light of an appropriate wavelength and intensity.

Following decontamination in accordance with the methods of this invention, the biological fluid can be stored or transfused in accordance with conventional practice. Alternatively, for fluids such as red cell preparations or platelet rich plasma, the decontaminated fluid can be centrifuged at a force sufficient to produce a pellet of the cellular components. The supernatant can be removed following centrifugation and the cells resuspended to reduce the concentration of residual photosensitizer and any reaction products.

The following examples are illustrative only and are not intended to limit the scope of the invention as defined by the appended claims. It will be apparent to those skilled in the art that various modifications and variations can be made in the methods of the present invention without departing from the spirit and scope of the invention.

All patents and publications referred to herein are expressly incorporated by reference.

### EXAMPLE 1

Compounds 1-26 in Table 2 were screened for virucidal and photohemolytic activity. Plasma containing red cells were oxygenated by gas overlay, leukodepleted by a 5 log₁₀ filter, suspended in Erythrosol to a hematocrit of 20%, and deliberately inoculated with extracellular VSV. Various concentrations of a compound were added to the oxygenated, leukodepleted cell suspension, and a 1 mm film of the suspension was subsequently illuminated for 2 minutes with 8.9 mW/cm² of red light (670 nm [peak intensity]±13 nm [half peak intensity]). Results in Table 2 correspond to compounds of Formula I.

TABLE 2*

| | |
|---|---|
| | Formula I |
| 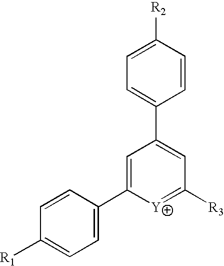 | |

| Compound # | Y | R₁ | R₂ | R₃ |
|---|---|---|---|---|
| 1 | O | N(CH₃)₂ | N(CH₃)₂ | CH₃ |
| 2 | S | N(CH₃)₂ | N(CH₃)₂ | CH₃ |
| 3 | Se | N(CH₃)₂ | N(CH₃)₂ | CH₃ |
| 4 | Te | N(CH₃)₂ | N(CH₃)₂ | CH₃ |
| 5 | S | N(CH₃)₂ | N(CH₃)₂ | φ |
| 6 | Se | N(CH₃)₂ | N(CH₃)₂ | φ |
| 7 | S | N(CH₃)₂ | N(CH₃)₂ | φ-N(CH₃)₂ |
| 8 | Se | N(CH₃)₂ | N(CH₃)₂ | φ-N(CH₃)₂ |
| 9 | Se | NH₂ | N(CH₃)₂ | φ-NH₂ |
| 10 | S | NH₂ | N(CH₃)₂ | φ |
| 11 | Se | NH₂ | N(CH₃)₂ | φ |
| 12 | S | NH₂ | morpholino | φ- morpholino |
| 13 | Se | NH₂ | morpholino | φ- morpholino |
| 14 | S | NH₂ | morpholino | φ-NH₂ |
| 15 | Se | NH₂ | morpholino | φ-NH₂ |
| 16 | S | NH₂ | H | φ- morpholino |
| 17 | Se | NH₂ | H | φ- morpholino |
| 18 | S | morpholino | morpholino | φ- morpholino |
| 19 | Se | morpholino | morpholino | φ- morpholino |
| 20 | S | NH₂ | N(CH₃)₂ | φ- morpholino |
| 21 | Se | NH₂ | N(CH₃)₂ | φ- morpholino |
| 22 | S | NH₂ | morpholino | φ |
| 23 | Se | NH₂ | morpholino | φ |
| 24 | Te | NH₂ | N(CH₃)₂ | φ |
| 25 | Te | N(CH₃)₂ | N(CH₃)₂ | φ-N(CH₃)₂ |
| 26 | Se | N(CH₃)₂ | H | φ |

*In Table 2, the symbol “φ” denotes a phenyl group.

TABLE 3

| | | Log₁₀ VSV | log₁₀ VSV | |
|---|---|---|---|---|
| | | inact. | inact. | |
| Compound | Concentration | (extra- | (intra- | Hemolysis (%) |
| # | (μM) | cellular) | cellular) | (day 42) |
| 1 | 20 | <0.5 | | |
| | 40 | <0.5 | | |
| | 80 | 1.3 | | |
| | 160 | 2.1 | | |
| 2 | 10 | 1.3 | | |
| | 20 | 2.2 | | |
| | 30 | 2.7 | | |
| | 60 | 4.8 | | |
| | 90 | 5.7 | | |
| | 160 | >7 | >5 | 0.45 |
| 3 | 10 | 0.9 | | |
| | 30 | 2.5 | | |
| | 60 | 4.2 | | |
| | 90 | 4.6 | | |
| | 120 | 5.6 | | |
| 4 | 20 | <0.5 | | |
| | 60 | <0.5 | | |
| | 120 | <0.5 | | |
| | 180 | <0.5 | | |
| | 240 | 0.5 | | |
| 5 | 1 | 3.1 | | 5.2 |
| | 5 | 5.9 | 2.3 | 5.1 |
| | 10 | 7.9 | 2.6 | 7.3 |
| | 15 | >7.9 | 3.7 | 6.4 |
| | 25 | >8.4 | 4.7 | 6.5 |
| 6 | 1 | | | |
| | 5 | | | |
| | 10 | >7.9 | 2.5 | 17.1 |
| | 15 | >8.1 | | 10.5 |
| | 25 | >8.0 | | 10.8 |
| 7 | 1 | 0.5 | | |
| | 5 | 1.0 | | |
| | 10 | 2.1 | | |
| | 15 | 3.0 | | |
| | 25 | 3.4 | | 3.2 (day 7) |
| 8 | 1 | 3.6 | | 2.4 (day 7) |
| | 5 | 7.4 | | 30.2 |
| | 10 | >8.3 | 4.7 | 42.2 |
| | 15 | >8.3 | | |
| | 25 | >8.3 | | |
| 9 | 1 | 0.6 | | |
| | 5 | 1.6 | | |
| | 10 | 2.3 | | |
| | 15 | 2.7 | | |
| | 25 | 3.3 | | |
| | 50 | 3.9 | | 0.83 |
| | 50 μM + | 6.9 | | 28.3 |
| | 2.2 J/cm² | | | |
| 10 | 1 | 0.7 | | |
| | 5 | 2.2 | | |
| | 10 | 3.8 | | |
| | 15 | 4.5 | | |
| | 25 | 5.6 | | 0.4 |
| | 40 | 5.1 | | 0.3 |
| | 40 μM + | 7.8 | 1.1 | 0.46 |
| | 2.2 J/cm² | | | |
| 11 | 1 | 1.9 | | |
| | 5 | 4.6 | | |
| | 10 | 6.2 | | 1.7 |
| | 15 | 7.4 | | 2.1 |
| | 25 | >8.2 | 2.4 | 2.5 |
| 12 | 25 | 2.2 | | |
| | 1 | 0.9 | | |
| | 5 | 1.5 | | |
| | 10 | 1.7 | | |
| | 15 | 2.0 | | |
| | 25 | 2.2 | 0.19 | |
| 13 | 1 | 0.9 | | |
| | 5 | 2.6 | | |
| | 10 | 3.3 | | |
| | 15 | 4 | | |
| | 25 | 4.5 | | |
| | 35 | 4.7 | | 0.6 |
| | 35 μM + | 7.0 | | 5.4 |
| | 2.2 J/cm² | | | |
| 14 | 1 | <0.5 | | |
| | 5 | <0.5 | | |
| | 10 | <0.5 | | |
| | 15 | <0.5 | | |
| | 25 | <0.5 | | |
| 15 | 1 | <0.5 | | |
| | 5 | <0.5 | | |
| | 10 | <0.5 | | |
| | 15 | <0.5 | | |
| | 25 | <0.5 | | |
| 16 | 1 | <0.5 | | |
| | 5 | <0.5 | | |
| | 10 | <0.5 | | |
| | 15 | <0.5 | | |
| | 25 | <0.5 | | |
| 17 | 1 | 0.7 | | |
| | 5 | 1.2 | | |
| | 10 | 1.7 | | |
| | 15 | 2.1 | | |
| | 25 | 1.7 | | |
| 18 | 1 | <0.5 | | |
| | 5 | <0.5 | | |
| | 10 | 0.7 | | |
| | 15 | 1.0 | | |
| | 25 | 1.2 | | 4.3 (day 7) |
| 19 | 1 | <0.5 | | |
| | 5 | <0.5 | | |
| | 10 | <0.5 | | |
| | 15 | <0.5 | | |
| | 25 | 1.3 | | 0.78 (day 7) |
| 20 | 1 | 0.8 | | |
| | 5 | 1.8 | | |
| | 10 | 2.5 | | |
| | 15 | 3.4 | | |
| | 25 | 4.0 | | |
| | 40 | 4.8 | | 0.9 |
| | 40 μM + | 7.1 | | 7.1 |
| | 2.2 J/cm² | | | |
| 21 | 1 | 1.6 | | |
| | 5 | 4.1 | | |
| | 10 | 5.3 | | 3.4 |
| | 15 | 5.7 | | 3.8 |
| | 25 | 7.8 | | 3.8 |
| 22 | 1 | <0.5 | | |
| | 5 | <0.5 | | |
| | 10 | <0.5 | | |
| | 15 | <0.5 | | |
| | 25 | 1.4 | | |
| 23 | 1 | 0.7 | | |
| | 5 | 1.8 | | |
| | 10 | 3.4 | | |
| | 15 | 4.0 | | |
| | 25 | 4.8 | | |
| | 30 | 5.7 | | 0.5 |
| | 30 + | 8.2 | | 3.5 |
| | 2.2 J/cm² | | | |
| 24 | 1 | <0.5 | | |
| | 5 | <0.5 | | |
| | 10 | <0.5 | | |
| | 15 | <0.5 | | |
| | 25 | 1.5 | | 4.9 |
| 25 | 1 | <0.5 | | 0.31 |
| | 5 | <0.5 | | 0.50 |
| | 10 | <0.5 | | 0.56 |
| | 15 | 1.1 | | 0.77 |
| | 25 | 2.1 | | 2.97 |
| 26 | 1 | <0.5 | | |
| | 5 | <0.5 | | |
| | 10 | <0.5 | | |
| | 15 | <0.5 | | |
| | 25 | <0.5 | | |

As shown in Table 3, compound 2, a diphenylpyrilium dye, inactivates>7 log₁₀ of extracellular VSV and >5 log₁₀ of intracellular VSV without causing undue hemolysis during 42 day 1-6° C. storage of red cells suspended in Erythrosol.

### EXAMPLE 2

Compound 2 was screened for bacteridcidal activity. Plasma containing red cells were oxygenated by gas overlay, leukodepleted by a 5 log₁₀ filter, suspended in Erythrosol to hematocrit of 20%, and inoculated with high levels of an organism to yield final bacterial counts ranging from 106 to 108 CFU/mL. Compound 2 was added to the deliberately contaminated, oxygenated, leukodepleted cell suspension to give a final concentration of 160 μM, and a 1 mm film of the suspension was subsequently illuminated for 2 minutes with 8.9 mW/cm² of red light (670 nm [peak intensity]±13 nm [half peak intensity]). Results are shown in Table 4.

TABLE 4

| Organism | log₁₀ inactivation |
|---|---|
| E. coli | >7.9 |
| S. marcescens | >6.9 |
| Y. enterocolitica | >7.5 |
| D. radiodurans | >7.9 |
| S. epidermidis | >7.9 |
| S. aureus | >6.8 |
| P. fluorescens | >7.7 |
| S. liquefaciens | >7.4 |

Both gram positive and gram negative organisms were inactivated by compound 2 and light to the limit of detection (<1 CFU/mL) using inoculula >6.8 log₁₀.

### EXAMPLE 3

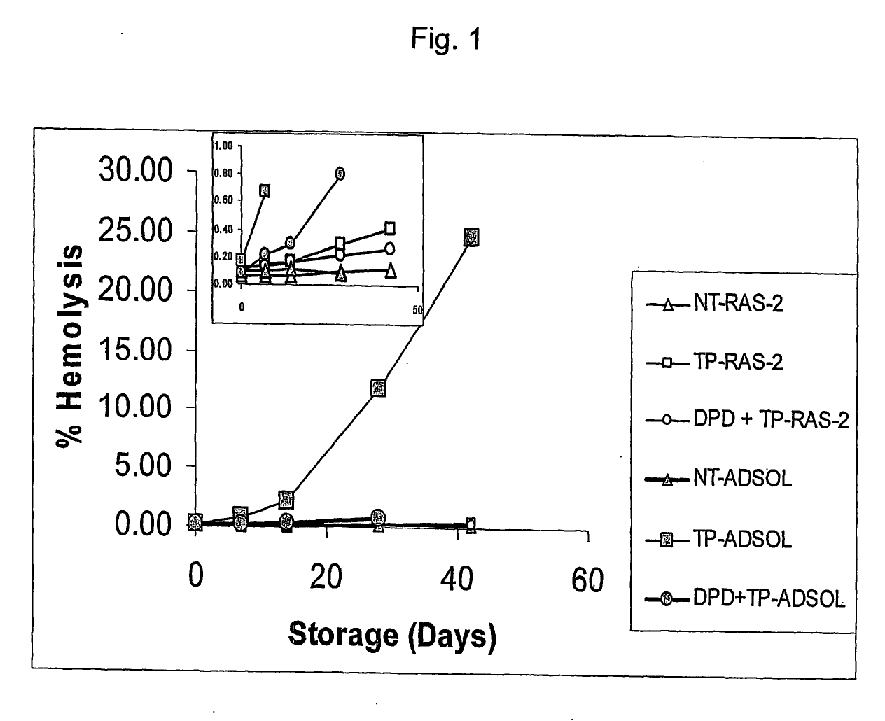

The effect of different additive solutions and dipyridamole on photoinduced hemolysis by compound 2 was studied. Plasma containing red cells were oxygenated by gas overlay, leukodepleted by a 5 log₁₀ filter, suspended in either Eiytlrosol or ADSOL additive solution to a hematocrit of 20%. Dipyridamole was added to some of the Erythrosol or ADSOL red cell suspensions to a final concentration of 200 μM. Compound 2 was then added to some of the Erythrosol or ADSOL red cell suspensions, some of which contained dipyridamole, to a final concentration of 160 μM. For suspensions containing compound 2, a 1 mm film of the suspension was subsequently illuminated for 2 minutes with 8.9 mW/cm² of red light (670 nm [peak intensity]±13 nm [half peak intensity]). Red cell suspensions were concentrated to 45% hematocrit by centrifugation. Untreated and phototreated red cell suspensions were stored for up to 42 days at 1-6° C. and assayed for hemolysis. Results are shown in FIG. 1. The data demonstrate, 1) that <1% hemolysis is observed in Erytlrosol containing RBC suspensions treated with compound 2 and light and 2) that less hemolysis is observed in RBC suspensions that contain dipyridamole than those that lack dipyridamole.

### EXAMPLE 4

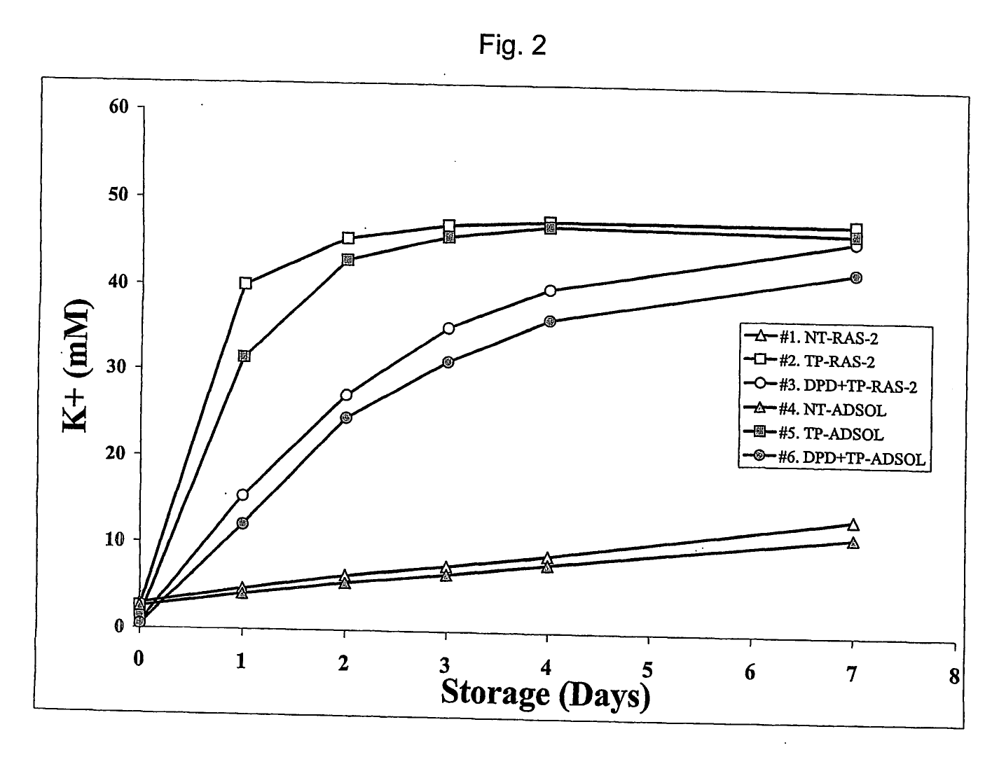

The effect of different additive solutions and dipyridamole on compound 2 photoinduced red cell potassium leakage was studied. Plasma containing red cells were oxygenated by gas overlay, leukodepleted by a 5 log₁₀ filter, and suspended in either Erythrosol or ADSOL additive solution to a hematocrit of 20%. Dipyridamole was added to some of the Erythrosol or ADSOL red cell suspensions to a final concentration of 200 μM. Compound 2 was then added to some of the Erythrosol or ADSOL red cell suspensions, some of which contained dipyridamole, to a final concentration of 160 μM. For suspensions containing compound 2, a 1 mm film of the suspension was subsequently illuminated for 2 minutes with 8.9 mW/cm² of red light (670 nm [peak intensity]±13 nm [half peak intensity]). Red cell suspensions were concentrated to 45% hematocrit by centrifugation. Untreated and phototreated red cell suspensions were stored for up to 42 days at 1-6° C. and assayed for extracellular potassium. Results are shown in FIG. 2. Data show that the addition of dipyridamole reduces potassium leakage of Erythrosol or ADSOL red cell suspensions during storage.

### EXAMPLE 5

Plasma containing red cells were oxygenated by gas overlay, leukodepleted by a 5 log₁₀ filter, and suspended in Erythrosol to a hematocrit of 20%, and deliberately inoculated with either intracellular or extracellular VSV. Dipyridamole was added to the oxygenated, leukodepleted red cell suspension at a final concentration of 200 μM. Compound 2 was then added to the red cell suspension at a final concentration of 160 μM, and a 1 mm film of the suspension was subsequently illuminated for 2 minutes with 8.9 mW/cm² of red light (670 nm [peak intensity]±13 nm [half peak intensity]). Samples were subsequently assayed for plaque forming ability. In the presence of dipyridamole, >7 log₁₀ inactivation of extracellular VSV and 4.0 log₁₀ of intracellular VSV was demonstrated.

### EXAMPLE 6

Red Blood Cell (RBC) preparation and oxygenation Packed RBCs were prepared from units of whole blood (500-50 mL) collected in 70 mL CDP in triple-pack container systems (PL146 primary container, Baxter Healthcare, Deerfield, Ill.) by the American Red Cross, Research Blood Department, Holland Laboratory for the Biomedical Sciences. Units were cooled to to 6° C. overnight, centrifuged at 1471×g for 4 minutes, and platelet-rich plasma and buffy coat were removed. The packed RBCs were diluted to an hematocrit (hct) of approximately 50% with cold Erythrosol (Hogman C F, Eriksson L, Gong J, Hogman A B, Vikholm K, Debrauwere J, Payrat J M, Stewart M. Half-strength citrate CPD combined with a new additive solution for improved storage of red blood cells suitable for clinical use, Vox Sang. 1993;65(4):271-8) or, when noted, with cold ADSOL (Baxter Healthcare); subsequently white cell reduced by using a filter (Leukotrap-SC RC, Pall Medical, East Hills, N.Y.); and oxygenated by adding 230 mL of a 60 to 40 percent O₂ to N₂ gas mixture to 150 mL of a RBC suspension in a 600 mL container (PL146 plastic, Baxter Healthcare) and by subsequent incubation for 30 minutes at 10 to 6° C. with agitation (orbital shaker, 100 r.p.m., 19-mm orbit, VWR Scientific, West Chester, Pa.). Oxygen levels were measured by use of a blood gas analyzer (Rapidalab 348, Bayer Corp., Medfield Mass.) and were routinely supersaturated with levels greater than 400 mm Hg.

TP Preparation

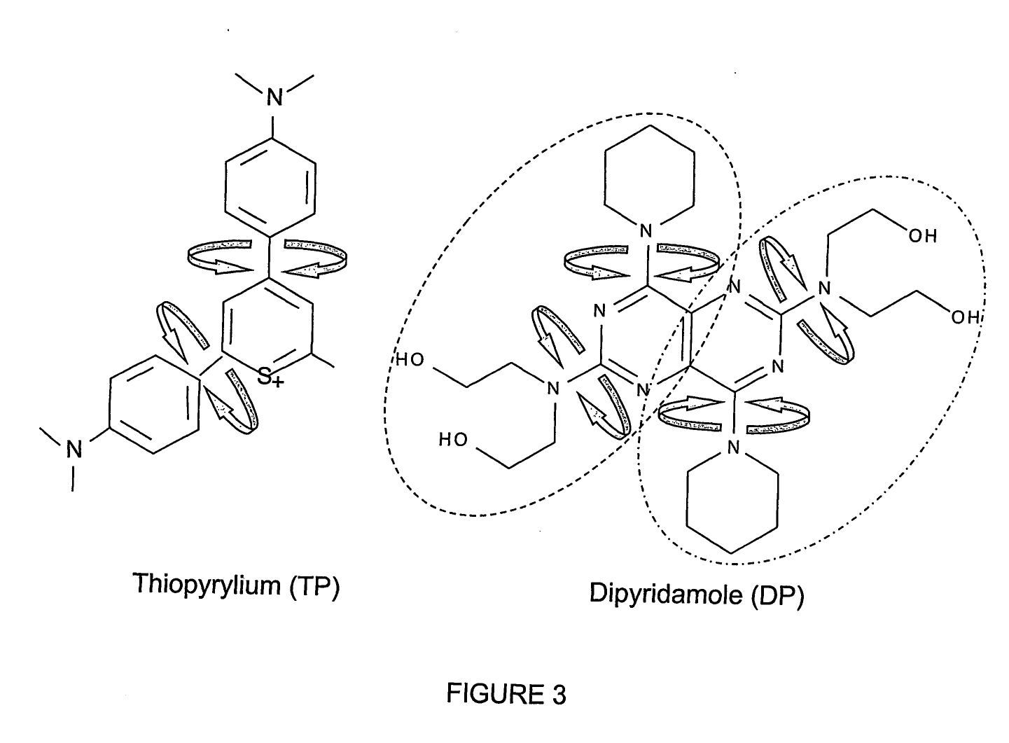

The structures of TP, or 2′,4′-bis(4-N,N-dimethylaminophenyl) 6′-methylthiopyrylium iodide, and DP are given in FIG. 3. TP was synthesized accord to the method described by Yamamoto and colleagues (Yamamoto N, Okamoto T, Miyazaki T, Kawaguchi M. Fluorescent stain containing pyrylium salt and fluorescent staining method of biological sample. U.S. Pat. No. 6,022,961). The dye was purified by medium pressure (100-psi) liquid chromatography. A gradient of methylene chloride:methanol (100:0-94:6) was used as an eluent for dye bound to silica gel (40μ, Scientific Absorbents, Atlanta, Ga.). The compound was homogeneous by thin layer chromatography. NMR analysis revealed the compound to be 90% pure, with 10% of the compound being the pyrylium precursor dye where an oxygen atom substitutes for sulfur in the central ring. Pathogen reduction experiments with purified pyrylium precursor revealed that the impurity possessed approximately one-third the photoactivity of TP at the same dye concentration (data not shown).

Addition of Virus or Bacteria and TP Phototreatment

We added stock cultures of extracellular and intracellular viruses or bacteria to oxygenated, white cell reduced, RBCs at approximately 50 percent hct. A 10 mM DP stock solution, a 500 μM freshly prepared stock solution of TP, each in Erythosol or ADSOL as noted in figures, and the appropriate additional additive solution were added sequentially to yield 200 μM DP and the desired TP concentration in the final 20% hct RBC suspension, which was utilized to improve light transmission, thereby maximizing pathogen reduction. The volume of the pathogen spike represented <10 percent of the total volume of the 20% hct RBC suspension. The suspension was thoroughly mixed and divided into 2 mL aliquots in polystyrene culture dishes (50 mm bottom diameter) to produce a 1 mm blood film. All treated and control samples contained DP and TP but control samples were not illuminated. We agitated culture dishes at room temperature on a horizontal reciprocal shaker (70 cycles/min) for 15 minutes in the dark prior to illumination.

Because RBC storage studies required greater volumes that virus or bacterial studies, 45 petri dishes containing 2 mL each of 20% hct RBCs with DP and TP were illuminated with agitation as described above, their contents pooled, concentrated to 45% hct by centrifugation and resuspension in the appropriate additive solution, and transferred to a 150-mL PL145 container to provide sufficient volume for RBC storage studies. RBC suspensions prepared at 45-percent hct that did not contain DP or TP and were not illuminated served as controls. In some red cell storage experiments, identically prepared RBC samples that contained TP but did not contain DP were illuminated to determine the protective effect of DP and are noted in the text and figures.

Illumination was carried out using a red LED source (Q-beam 2001-MED, Quantum Devices, Inc., Barneveld, Wis.), which emitted 670 (peak intensity)±13 nm (half peak intensity) light with fluence rates adjustable up to 9.0 mW/cm². Fluence rates were measured by use of a handheld laser power meter with a silicon cell sensor (Edmunds Industrial Optics, Barrington, N.J.). All phototreated samples were exposed 2 minutes to the 9.0 mW/cm² source, corresponding to a 1.1 J/cm² light exposure.

Virus Assays

Source of virus, bacteria and infected host cells. VSV was provided by Med Lieu (Hyland Diagnostics, Duarte, Calif.). BVDV was purchased from the American Type Culture Collection, Manassas, Va.). PRV was provided by Shirley Mieka (American Red Cross, Rockville, Md.). DHBV, HIV-1 IIIB, and an HIV-1 infected HUT 78 permissive B-cell line, BP-1 (originally isolated by Bernard Poiesz using the method of Federico M, Titti F, Butto S, Orecchia A, Carlini F, Taddeo B, et al., Biologic and molecular characterization of producer and nonproducer clones from HUT-78 cells infected with a patient IV isolate, AIDS Res Hum Retroviruses, 1989;5:385-96), was obtained from M. Khalid Ijaz (MicroBioTest, Sterling, Va.). Clinical strains of E. coli, P. fluorescens were provided by Joseph Campos (Childrens' National Medical Center, Washington, D.C.). Clinical strain of S. marcescens and Y. enterocolitica (serotype O:3) was provided by Vince Piscitelli (Yale New Haven Hospital, New Haven, Conn.). S. epidermidis (ATCC #1228), S. aureus (ATCC #27217), and S. liquifaciens (ATCC #27529) were purchased from the American Type Culture Collection, Manassas, Va.

Mammalian virus assays. We propagated VERO (isolated from African green monkey kidney, CCL81, ATCC) and MDBK (CRL6071, ATCC) cells in medium (RPMI 1640 supplemented with glutamine, Biofluids, Rockville, Md.) supplemented with 10-percent bovine serum. Cells were seeded into six-well culture plates and allowed to grow to confluency. Control and phototreated samples were serially diluted 10-fold, plated onto confluent VERO (for VSV and PRV) or MDBK (for BVDV) cell monolayers, and incubated for 1 hour with gentle rocking at 37° C. for virus adsorption to cells. The inoculum was removed by aspiration and washed with PBS, a semi-liquid agar layer (0.2-percent) was added to each well and infected monolayers were incubated at 37° C. in air containing 5-percent CO₂. Incubation periods were: VSV, 1 day; PRV, 2 to 3 days; BVDV, 5 to 6 days. After incubation, the agar layer was removed by aspiration and the monolayer was stained with 0.1-percent crystal violet in ethanol for at least 15 minutes. The stain was removed by aspiration, the plates were washed with water, and the plaques enumerated.

The isolation and culture of primary duck hepatocytes from <1-week-old seronegative White Pekin ducklings (Anas domesticus) and DHBV immunofluorescence assay was performed by MicroBioTest (Sterling, Va.) according to a previously published procedure (Wagner S J, Skripchenko A, Pugh J C, Suchmann D B, Ijaz M, Duck hepatitis B photoinactivation by dimethylmethylene blue in RBC suspensions, Transfusion, 2001;41:1154-8). Treated and control DHBV-spiked RBC samples were serial diluted 1 in 10 in L-15 medium (BioFluids) and 1-mL volumes were subsequently inoculated in primary duck hepatocytes monolayers in quadruplicate. The infected cultures were incubated at 37° C. with 5-percent CO₂ overnight for virus attachment and entry. The inoculum was then removed, cell monolayers were washed once with complete L-15 medium to remove excess RBCs, and then each well was overlaid with approximately 2 mL of fresh L-15 medium. Infected monolayers were incubated an additional 6 to 7 days at 37° C., with media changes every 2 days. After incubation, the medium was removed by aspiration, monolayers were washed with PBS and removed by aspiration, and monolayers were subsequently fixed by incubation with 1 to 2 mL of −20° C. ethanol for 2 hours at 4° C. The ethanol was removed by aspiration, washed with PBS, and incubated at room temperature for at least two hours with 0.25 mL of a 1 in 2-mL dilution of DHBV MoAb directed against the pre-S domain of the DHBV envelope (Pugh J C, Di Q, Mason W S, Simmons H, Susceptibility to duck hepatitis B virus infection is associated with the presence of cell surface receptor sites that efficiently bind virus particles, J Virol 1995;69:4814-22). The antibody was removed by aspiration, washed with PBS, aspirated, and incubated for 2 hours at room temperature with 0.25 mL of a 1-in-200 dilution of goat anti-mouse IgG-FITC conjugate (Jackson Immuno-Research Laboratories, West Grove, Pa.). The secondary antibodies were removed by aspiration, and the fluorescence-stained monolayer was washed with PBS and aspirated. Monolayers were examined by UV light fluorescent microscopy (Diaphot, Nikon, Columbia, Md.) and were scored positive if wells contained one or more DHBV surface-antigen-positive hepatocytes. Virus titers were determined by the median tissue culture infective dose method (Reed L J, Muench H A, A simple method of estimating fifty percent end points, Am J Hyg 1938;27:493-7).

Titration of extracellular and intracellular HIV-1 was carried out by MicroBioTest, Sterling, Va. Control and phototreated RBCs containing extracellular or intracellular HIV-1 were serially diluted 10-fold in RPMI1640 supplemented with glutamine (ATCC, Manassas, Va.) and containing 10% fetal bovine serum (Invitrogen, Calif.), and 0.5 mL of each dilution of control or phototreated sample was transferred into 24-well plates (Corning, Acton, Mass.) in quadruplicate. To each of these wells was added 1 mL of a T-cell lymphoblastic host cell line, CCRF-CEM, for coculture (Yamada O, Hattori N, Kurimura T, Kita M, Kishida T, Inhibition of growth of HV by human natural interferon in vitro, AIDS Res Hum Retroviruses, 1988;4:287-94). Virus and host cells were incubated at 37° C. in 5% CO₂ in air for 18-24 hours for virus adsorption, and then one-half of the cell suspension was replaced with fresh medium. Infected cells were incubated an additional 3-4 weeks with a replacement of one-half of the volume of the supernatant with fresh medium 3 times per week. After 9-12 additions of fresh media, we assayed culture fluid by HIV-1 p24 antigen enzyme-linked immunosorbent assay (Zepto Matrix, Buffalo, N.Y.).

Bacterial Assays

We prepared fresh overnight cultures of bacteria by inoculating single-colony isolated into Luria broth (Becton Dickinson, Cockeysville, Md.). Cultures were incubated under aerobic conditions at 30 or 37° C., depending on the strain. Following inoculation into RBC suspensions, bacterial counts were determined in phototreated and control samples by 1-in-100 serial dilution of fully mixed samples in unbuffered saline, adding either 0.1 or 1.0 mL of the diluted or neat suspension, respectively, to 3 mL of 0.8-percent molten agar (43° C.) and pouring the molten agar over Luria broth agar plates. We counted colonies after incubation for 24-72 hours at 30 or 37° C., with time and incubation temperature depending on the strain. Colonies were counted from all plates that contained between 1 and 750 colonies per plate.

RBEC Assays

We assayed for ATP using the method of Beutler (Red cell metabolism. A manual of biochemical methods. Third Edition, by Earnest Beutler, Grune & Stratton Inc., Orlando, Fla., 1984). Supernatant Hb was determined by the tetramethylbenzidine method (Procedure No. 527, Sigma) (Standefer J C, Vanderagt D, Use of tetramethylbenzidine in plasma hemoglobin assay, Clin Chem, 1977;23:749-51). Total hemoglobin was determined by an automated cell counter (Cell Dyn 3700, Abbott Laboratories, Abbott Park, Ill.). We measured extracellular potassium, pH, lactate and glucose using blood gas analyzers (RapidLab 348 or RapidLab 860, Bayer Corp). For morphology studies, we fixed RBCs in glutaraldehyde and scored 200 cells by cell type: discocyte, 1.0; echinocyte I, 0.8; echinocyte II, 0.6; echinocyte III, 0.4; echinospherocyte 0.2; and spherocyte, 0.0 (Usry R T, Moore G L, Manalo F W, Morphology of stored, rejuvenated human eiythrocytes, Vox Sang, 1975;28:176-83).

Statistics

Determination of means and standard deviation of experimental values, the performance of two-tailed, tests with Welch's correction, and linear regressions were carried out by using standard software (Instat, GraphPad Software, San Diego, Calif.). A value of p<0.05 was considered significant. In a number of phototreated samples, no plaques, infected cells or bacteria were visible in wells or culture dishes containing either diluted or undiluted samples. In these circumstances, the extent of inactivation was calculated with the assumption that one plaque or colony was observed, and the extent of pathogen inactivation was reported as greater than the calculated value.

Results

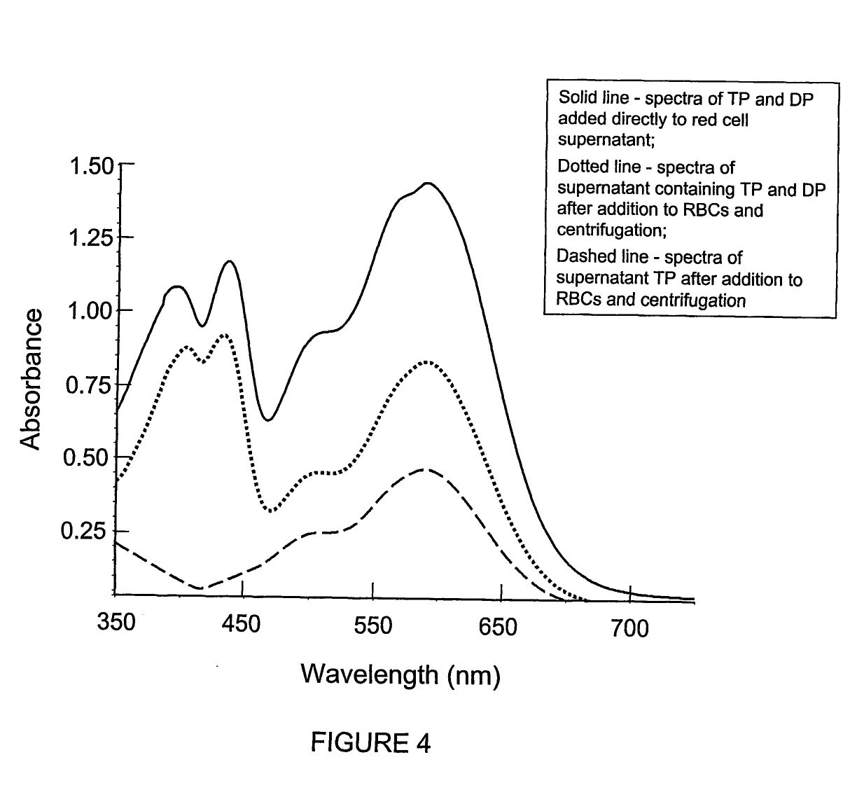

RBC binding studies. A spectroscopic assay was developed to measure the effect of DP on TP binding to RBCs suspended in Erythrosol. TP was added to freshly prepared 20-percent hct RBCs to yield a final concentration of 160 μM in suspensions containing or lacking 200 μM DP. Following centrifugation of RBCs, supernatant spectra were compared to spectra of 160 μM TP added directly to supernatant. Results are given in FIG. 4. Addition of TP to RBC suspensions lacking DP results in a spectra (dashed line) whose 620 nm TP peak is reduced 70-percent from the peak of the spectra representing TP added directly to supernatant (solid line). The reduction of the TP peak when the dye is incubated with RBCs suggests that a substantial fraction of the dye is bound to cells. Addition of TP to RBCs containing DP results in supernatant spectra (dotted line) whose peak is partially restored to that of the dye added directly to supernatant. The partial restoration of the supernatant TP peak intensity in RBCs containing DP suggests that DP blocks TP binding of some (approximately 36-percent), but not all sites in RBCs.

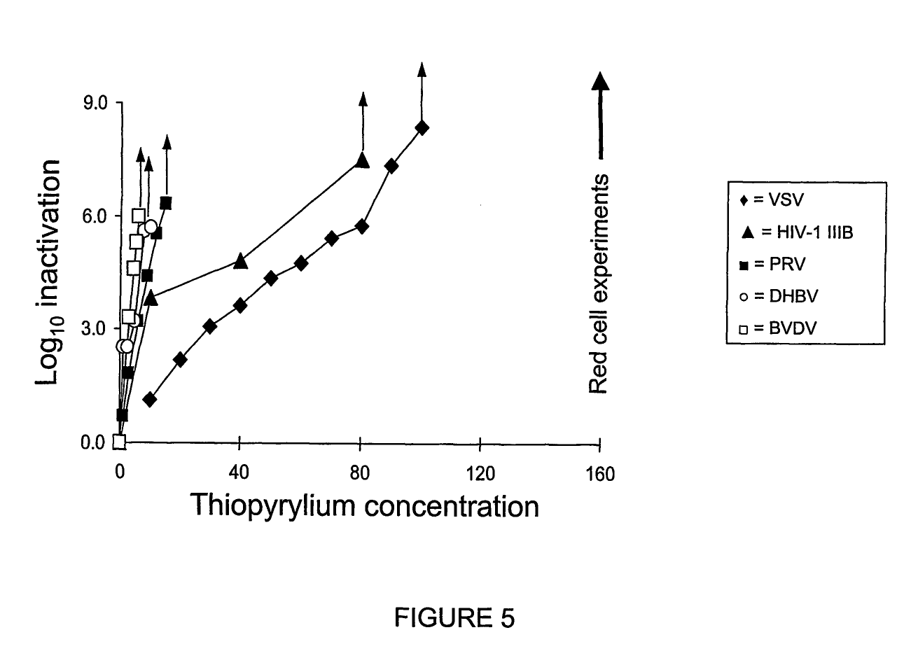

Virus inactivation studies. Virus inactivation experiments were performed in 20-percent hct RBCs suspended in Erythrosol and containing 200 μM DP. The extent of inactivation using 1.1 J/cm² light was measured as a function of TP dose. In general, the log₁₀ inactivation of each virus varied linearly with TP concentration (FIG. 5). Sensitivities to inactivation varied greatly among different viruses. Phototreatment of RBC suspensions containing DP resulted in >8.4 log₁₀ of extracelluar VSV at 100 μM TP, >7.5 log₁₀ extracellular HIV at 80 μM TP, 6.2±0.1 log₁₀ intracellular HIV at 80 μM TP, >6.3 log₁₀ extracellular PRV at 15 μM TP, >5.8 log₁₀ extracellular DHBV at 10 μM TP, and >6 log₁₀ extracellular BVDV at 4 μM TP. With 100 μM TP, all tested viruses were inactivated to the limit of assay detection. The vertical line in FIG. 5 represents the 160 μM TP concentration used to assess RBC storage properties following phototreatment.

Bacterial inactivation studies. Photoinactivation of gram positive and gram negative organisms was studied in experiments which utilized 100 μM TP and 200 μM DP in 20% hct RBCs suspended in Erythrosol. Results are given in Table 5. TP phototreatment resulted in >4 log₁₀ inactivation of all tested organisms and >6 log₁₀ inactivation of 5 of 7 tested organisms.

TABLE 5
Bacterial inactivation by 100 μM TP,
200 μM DP and 1.1 J/cm² red light

| Organism | log₁₀ Inactivation |
|---|---|
| S. epidermidis | >7.6 |
| S. aureus | >6.3 |
| Y. enterocolitica | 5.9 ± 0.6 |
| E. coli | 7.1 ± 0.5 |
| P. fluorescens | 5.4 ± 1.0 |
| S. liquifaciens | >7.1 |
| S. marcescens | 6.2 ± 1.0 |

n = 4

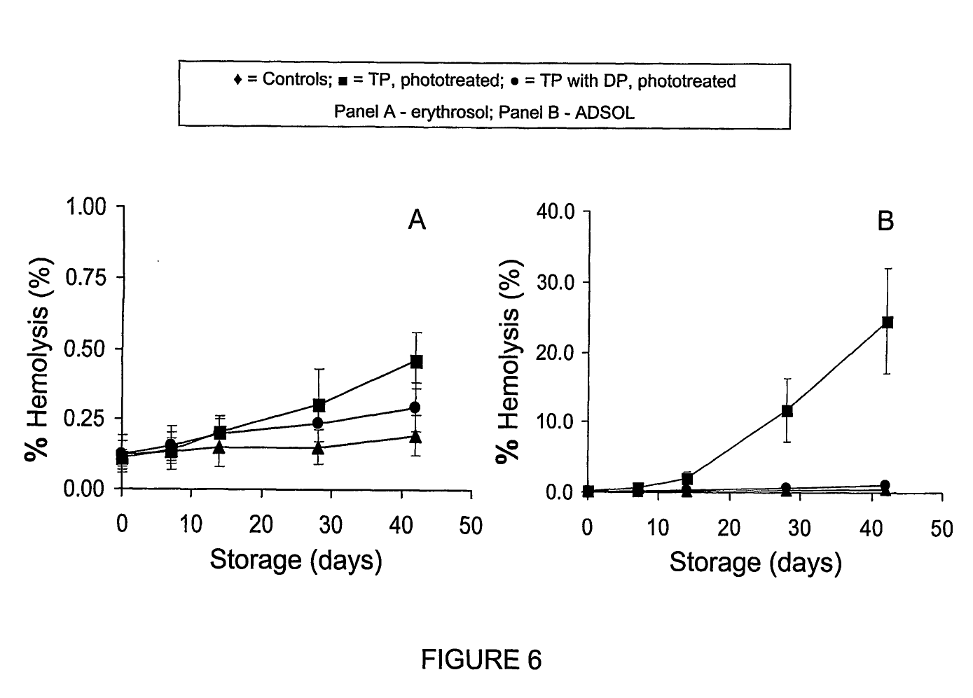

RBC storage studies. FIG. 6 shows the effect of dipyridamole and choice of additive solution on hemolysis following the storage of phototreated RBCs (160 μM TP and 1.1 J/cm² light). In the presence of 200 μM DP, TP phototreated RBC suspended in Erythrosol (panel A) had low levels (0.29%) of hemolysis following 42 day 1-6° C. storage, but were elevated compared to controls (0.19%) (p<0.05, n=10). Much less hemolysis was observed in phototreated RBCs suspended in Erythrosol and lacking DP than those stored in ADSOL and lacking DP (0.46±0.1 vs. 24.56±7.57 at day 42). Although DP significantly (p<0.05) reduced photoinduced hemolysis of RBCs suspended in both additive solutions, its effect was much more evident in cells suspended in ADSOL than those suspended in Ezythrosol.

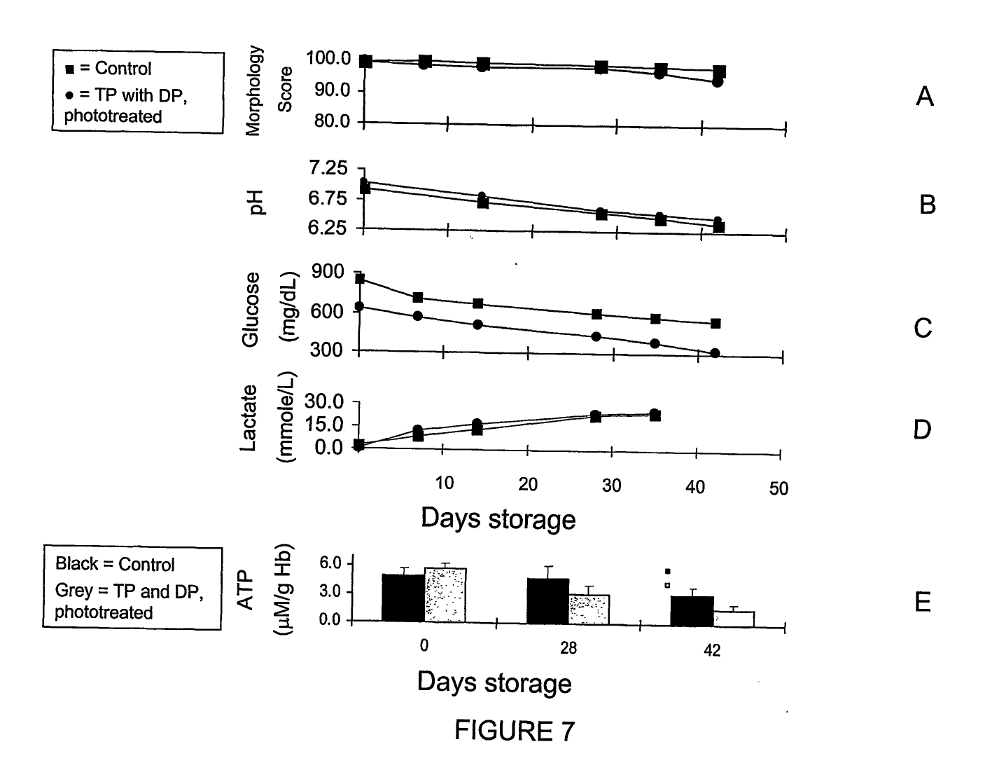

The effect of 160 μM TP, 200 μM DP and 1.1 J/cm² light on morphology score, pH, glucose utilization, lactate production, and ATP levels of RBCs suspended in Erytrosol is given in FIG. 7, panels A through E, respectively. TP phototreatment resulted in acceptable but significantly (p<0.05) different morphology scores (94.5±1.5 vs. 98.3±0.7, n=4) and extracellular pHs (6.48±0.07 vs. 6.38±0.06, n=10) at 42 days of storage. Although glucose levels were greater in phototreated samples because of their dilution (to 20-percent hct) and resuspension with Erytlrosol (to 45-percent hct), similar (p>0.05) glucose utilization rates (6.20±0.97 vs. 7.08±0.31 mM/day, n=4), and lactose production (26.7±1.9 vs. 28.2±1.0 mM on day 42, n=4) were observed in treated and control samples. ATP levels of phototreated samples declined with storage more rapidly than controls (2.63±0.07 vs. 3.61±0.22 μmole/g Hb, day 42, p<0.05, n=4).

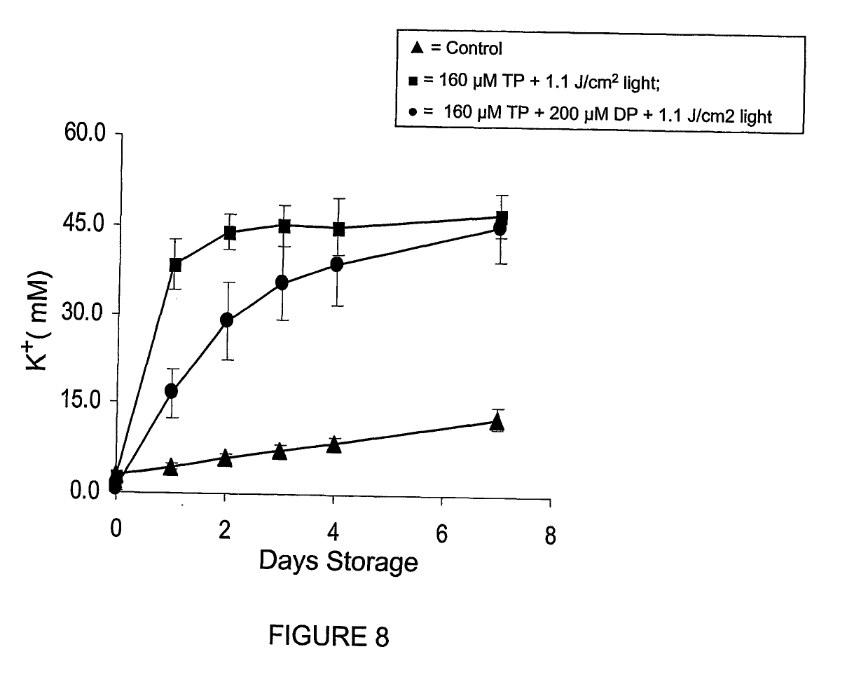

FIG. 8 shows the effect of DP on potassium release from RBCs suspended in Erythrosol and treated with 160 μM TP and 1.1 J/cm² light. Potassium leakage from phototreated RBCs is very rapid, with 38.4±4.4 mM potassium released to the supernatant after one day of storage compared to 4.4±0.4 mM in controls (p<0.05, n=10). In the presence of DP, potassium efflux from phototreated RBCs is partially inhibited, with 16.6±4.2 mM in the supernatant after one day of storage (p<0.05 compared to either control or phototreated samples lacking DP, n=10). Very similar results were obtained in control and phototreated RBCs suspended in ADSOL (data not shown).

What is claimed is:

1. A method for decontaminating a biological fluid comprising: (a) contacting a biological fluid with a virucidal effective amount of a diphenylpyrilium dye; and (b) irradiating the resulting mixture with light of about 560 to about 800 nm to achieve a virucidal effect.

2. The method of claim 1, wherein the biological fluid is selected from the group consisting of: blood, cellular blood components, liquid blood components; and mixtures of cellular and liquid blood components.

3. The method of claim 1, wherein the diphenylpyrilium dye is a compound of the formula: 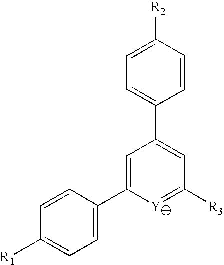
wherein:
Y is S or Se;
R₁ and R₂ are independently selected from the group consisting of amino, allylamino, and alkoxyamino; and
R₃ is alkyl, alkoxy, or aryl, arylamino, and arylalkoxyamino.

4. The method of claim 1 wherein the virucidal effective amount is that sufficient to achieve a virus kill capacity of at least about 6.0 log₁₀ extracellular virus in the biological fluid.

5. The method of claim 1 wherein the virucidal effective amount is that sufficient to achieve a virus kill capacity of at least about 3.5 log₁₀ intracellular virus in the biological fluid.

6. The method of claim 1 wherein the step of contacting a biological fluid with a virucidal effective amount of a diphenylpyrilium dye further comprises the addition of dipyridamole.

7. The method of claim 5 wherein the decontamination causes less than about 3% hemolysis in a red blood cell containing biological fluid upon exposure to the diphenylpyrilium dye for about 42 days at about 1-6° C.

8. A method for decontaminating a biological fluid comprising: (a) adding to the biological fluid dipyridamole and a virucidal effective amount of a diphenylpyrilium dye of the formula: 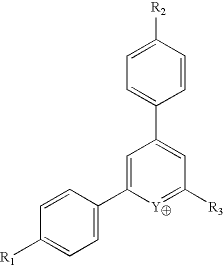
wherein Y is S or Se; R₁ and R₂ are independently amino, methylamino, or dimethylamino; and R₃ is alkyl of 1-6 carbons or phenyl; and
(b) irradiating the resulting mixture with at least about 0.025 J/cm² of light of about 560 nm to about 800 nm to decontaminate said biological fluid.

9. The method of claim 8 wherein the dipyridamole is added to the biological fluid to a concentration of at least about 100 μM.

10. The method of claim 8 wherein the irradiation step comprises irradiating said biological fluid with about 0.1 to about 1.0 J/cm² of light of about 590 nm to about 640 nm wavelength.

11. The method of claim 8 wherein Y is S; R₁ and R₂ are both dimethylamino; and R₃ is methyl.

12. The method of claim 11 wherein the virucidal effective amount is that sufficient to achieve at least about a 30 μM diphenylpyrilium concentration in the biological fluid.

13. The method of claim 11 wherein the virucidal effective amount is that sufficient to achieve at least about a 160 μM diphenylpyrilium concentration in the biological fluid.

14. The method of claim 8 wherein the virucidal effective amount constitutes sufficient diphenylpyrilium to achieve a virus kill capacity corresponding to at least about 7.0 log₁₀ extracellular VSV in the biological fluid.

15. The method of claim 8 wherein the virucidal effective amount constitutes sufficient diphenylpyrilium to achieve a virus kill capacity corresponding to at least about 3.5 log₁₀ intracellular VSV in the biological fluid.

16. The method of claim 8 wherein the biological fluid is selected from the group consisting of: cellular blood components, liquid blood components; and mixtures of cellular and liquid blood components.

17. The method of claim 8 wherein the decontamination inactivates gram positive and gram negative organisms to a level of greater than 6 log₁₀.

18. The method of claim 8 wherein the decontaminated fluid experiences less than about 1% hemolysis over a period of about 42 days following decontamination at about 1-6° C.

19. A method for decontaminating a biological fluid comprising:
(a) adding 2′,4′-bis(4-N,N-dimethylaminophenyl) 6′methylthiopyrilium iodide to the biological fluid to a concentration of about 50 to about 300 μM;
(b) adding dipyridamole to the biological fluid to a concentration of about 100 to about 300 μM; and
(c) irradiating the resulting biological fluid with light of about 560 to about 800 nm.
